(19) United States
(12) Patent Application Publication
(10) Pub. No.: US 2003/0208221 A1
(43) Pub. Date: Nov. 6, 2003

(54) Catheter with a coiled support member

(76) Inventor: Fozan El-Nounou, Sunnyvale, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/137,526

(22) Filed: May 2, 2002

(51) Int. Cl. A61M 29/00

(52) U.S. Cl. 606/191

(57) ABSTRACT

A catheter having an elongated shaft with a proximal shaft section and a distal shaft section, and having a support member with a proximal end within the proximal shaft section, a distal end within the distal shaft section, and at least a section which is coiled. In a presently preferred embodiment, the support member is a wire, and preferably a solid wire formed of a metal such as a stainless steel or super elastic alloy such as a nickel-titanium (Nitinol) alloy. In a presently preferred embodiment, the proximal shaft section comprises a high strength tubular member, such as a metallic tubular member. The catheter of the invention is highly pushable, flexible, trackable and kink resistant with the support member extending from the distal end of the relatively stiff proximal shaft section at the junction between the proximal and distal shaft sections.

### BACKGROUND OF THE INVENTION

This invention generally relates to catheters, and particularly intravascular catheters for use in percutaneous transluminal coronary angioplasty (PTCA) or for the delivery of stents.

In percutaneous transluminal coronary angioplasty (PTCA) procedures a guiding catheter is advanced in the patient's vasculature until the distal tip of the guiding catheter is seated in the ostium of a desired coronary artery. A guidewire is first advanced out of the distal end of the guiding catheter into the patient's coronary artery until the distal end of the guidewire crosses a lesion to be dilated. A dilatation catheter, having an inflatable balloon on the distal portion thereof, is advanced into the patient's coronary anatomy over the previously introduced guidewire until the balloon of the dilatation catheter is properly positioned across the lesion. Once properly positioned, the dilatation balloon is inflated with inflation fluid one or more times to a predetermined size at relatively high pressures so that the stenosis is compressed against the arterial wall and the wall expanded to open up the vascular passageway. Generally, the inflated diameter of the balloon is approximately the same diameter as the native diameter of the body lumen being dilated so as to complete the dilatation but not overexpand the artery wall. After the balloon is finally deflated, blood flow resumes through the dilated artery and the dilatation catheter and the guidewire can be removed therefrom.

In such angioplasty procedures, there may be restenosis of the artery, i.e. reformation of the arterial blockage, which necessitates either another angioplasty procedure, or some other method of repairing or strengthening the dilated area. To reduce the restenosis rate of angioplasty alone and to strengthen the dilated area, physicians now normally implant an intravascular prosthesis, generally called a stent, inside the artery at the site of the lesion. Stents may also be used to repair vessels having an intimal flap or dissection or to generally strengthen a weakened section of a vessel or to maintain its patency. Stents are usually delivered to a desired location within a coronary artery in a contracted condition on a balloon of a catheter which is similar in many respects to a balloon angioplasty catheter, and expanded within the patient's artery to a larger diameter by expansion of the balloon. The balloon is deflated to remove the catheter and the stent left in place within the artery at the site of the dilated lesion. See for example, U.S. Pat. No. 5,507,768 (Lau et al.)and U.S. Pat. No. 5,458,615 (Klemm et al.), which are incorporated herein by reference.

An essential step in effectively performing a PTCA procedure is properly positioning the balloon catheter at a desired location within the coronary artery. To properly position the balloon at the stenosed region, the catheter must have good pushability and flexibility to be readily advanceable within the tortuous anatomy of the patient's vasculature. Conventional balloon catheters for intravascular procedures, such as angioplasty and stent delivery, frequently have relatively a stiff proximal shaft section to facilitate advancement of the catheter within the patient's body lumen and a relatively flexible distal shaft section to facilitate passage through tortuous anatomy such as distal coronary and neurological arteries without damage to the luminal wall. Typically, there is an intermediate section or junction between the relatively stiff proximal shaft section and the relatively flexible distal shaft section which provides a transition between the proximal shaft section and less flexible than the distal shaft section.

A variety of intermediate section or junction designs have been utilized to provide a relatively smooth transition between the stiff proximal shaft section and the flexible distal shaft section. However, it has been difficult to develop a catheter design with an intermediate catheter shaft junction which provides a smooth transition and improved flexibility and pushability, and which is also leak free when utilizing high pressure inflation fluid to inflate the balloon on the distal shaft section of the catheter for dilatation or stent deployment. What has been needed is a catheter which is highly trackable within the patient's anatomy, with improved flexibility and pushability.

### SUMMARY OF THE INVENTION

The invention is directed to a catheter having an elongated shaft with a proximal shaft section and a distal shaft section, and having a support member with a proximal end within the proximal shaft section, a distal end within the distal shaft section, and at least a section which is coiled. In a presently preferred embodiment, the support member has at least a section which is not coiled.

In a presently preferred embodiment, the catheter is a balloon catheter. The balloon catheter of the invention may comprise a variety of suitable balloon catheters, including coronary and peripheral dilatation catheters, stent delivery catheters, drug delivery catheters, and the like. A balloon catheter of the invention generally comprises an elongated shaft with an inflation lumen, a guidewire receiving lumen, a proximal shaft section defining a proximal portion of the inflation lumen, and a distal shaft section defining a distal portion of the inflation lumen, with an inflatable balloon on the distal shaft section. At least part of the guidewire receiving lumen extends within the distal shaft section to a guidewire distal port in the distal end thereof.

In one embodiment, the catheter is a rapid exchange type catheter having a guidewire proximal port in the distal shaft section spaced a relatively short distance proximally from the guidewire distal port and a relatively long distance from the proximal end of the catheter shaft, a guidewire distal port at the distal end of the catheter, and a relatively short guidewire receiving lumen extending between the proximal and distal guidewire ports in the distal shaft section. In an alternative embodiment, the catheter is an over-the-wire type catheter having an elongated shaft with proximal and distal ends, a guidewire port in the proximal end, a guidewire port in the distal end, and a guidewire lumen extending therein from the distal end to the proximal end of the catheter shaft.

In a presently preferred embodiment, the proximal shaft section comprises a high strength tubular member, such as a metallic tubular member commonly referred to as a hypotube, with the support member in a distal end thereof. The metallic tubular member is typically formed of stainless steel, although a variety of suitable high strength materials may be used such as a nickel-titanium (Nitinol)alloy, MP35N, and Elgiloy, and including polymeric materials such as polyetheretherketone (PEEK), polyamides, and reinforced polymers, or other suitable high strength materials from which small diameter tubing can be readily formed.

In a presently preferred embodiment, the support member is a wire, and preferably a solid wire formed of a metal including a stainless steel or super elastic alloy such a nickel-titanium (Nitinol) alloy. The support member provides kink resistance at the distal end of the relatively stiff proximal shaft section and a smooth transition to the more flexible distal shaft section. For example, the support member provides an improved stiffness transition at the junction between a relatively stiff high strength proximal tubular member and relatively flexible distal section. Additionally, in the embodiment in which the catheter is a rapid exchange catheter, the support member provides increased rigidity at the rapid exchange junction weakened by the presence of the rapid exchange guidewire proximal port.

In a presently preferred embodiment, the support member has a noncoiled proximal section which is proximal to the coiled section, and which is at least inpart within the proximal shaft section. The term “noncoiled” should be understood to refer to sections extending distally in a primarily longitudinally oriented direction and not helically in a spiraling configuration. In one embodiment, the noncoiled section is substantially straight, i.e., extends in a line which is straight within normal manufacturing tolerances. Preferably, the support member comprises a coiled section located between a noncoiled proximal section and a noncoiled distal section. The coiled section is preferably has tightly wound coils which are stacked together, to provide excellent transmission of force for improved catheter pushability. The support member is typically secured to a portion of the shaft. In one embodiment, the noncoiled proximal section is secured to an inner surface of the proximal shaft section, as for example by adhesive bonding, or by welding, soldering, or crimping.

The catheter of the invention is highly pushable, flexible, trackable and kink resistant due to the support member extending from the distal end of the relatively stiff proximal shaft section at the junction between the proximal and distal shaft sections. A junction design of the invention provides an improved transition between the proximal shaft section and the more flexible distal shaft section, for improved kink resistance. Thus, the flexible and pushable distal shaft section provides a catheter with excellent trackability, and allows easy advancement over a guidewire and maneuvering within the patient's tortuous anatomy, to position the operative portion of the catheter at a desired location within the patient. Moreover, the catheter has a low profile, with a large inflation lumen for improved inflation/deflation times. These and other advantages of the invention will become more apparent from the following detailed description of the invention and the accompanying exemplary drawings.

### BRIEF DESCRIPTION OF THE DRAWINGS

FIG. 1 is an elevational view, partially in section, of a catheter which embodies features of the invention, having a rapid exchange distal guidewire lumen.

FIG. 2 is a transverse cross sectional view of the catheter shown in FIG. 1, taken along line 2-2.

FIG. 3 is a transverse cross sectional view of the catheter shown in FIG. 1, taken along line 3-3.

FIG. 4 is a transverse cross sectional view of the catheter shown in FIG. 1, taken along line 4-4.

FIG. 5 is a transverse cross sectional view of the catheter shown in FIG. 1, taken along line 5-5.

FIG. 6 is a transverse cross sectional view of the catheter shown in FIG. 1, taken along line 6-6.

FIG. 7 is an elevational view, partially in section, of an alternative embodiment of a catheter which embodies features of the invention, having a guidewire lumen extending the length of the catheter and the support member proximal end in a proximal outer tubular member.

FIG. 8 is a transverse cross sectional view of the catheter shown in FIG. 7, taken along line 8-8.

FIG. 9 is an elevational view, partially in section, of an alternative embodiment of a catheter which embodies features of the invention, having a guidewire lumen extending the length of the catheter and the support member proximal end in a proximal inner tubular member.

FIG. 10 is a transverse cross sectional view of the catheter shown in FIG. 9, taken along line 10-10.

### DETAILED DESCRIPTION OF THE INVENTION

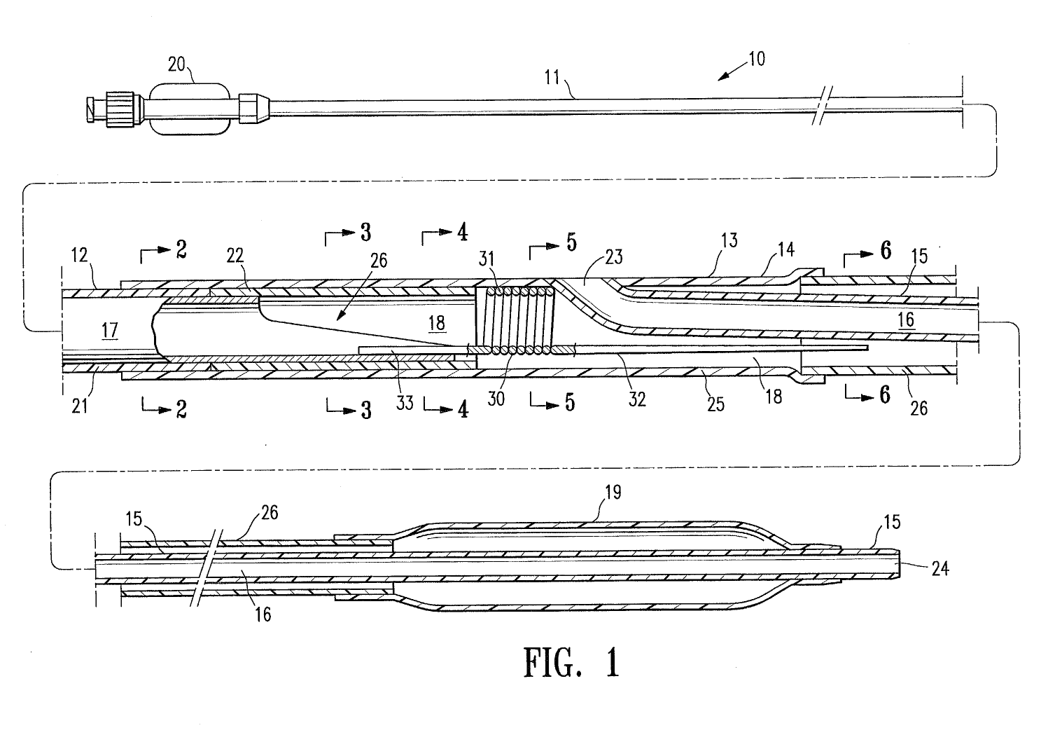

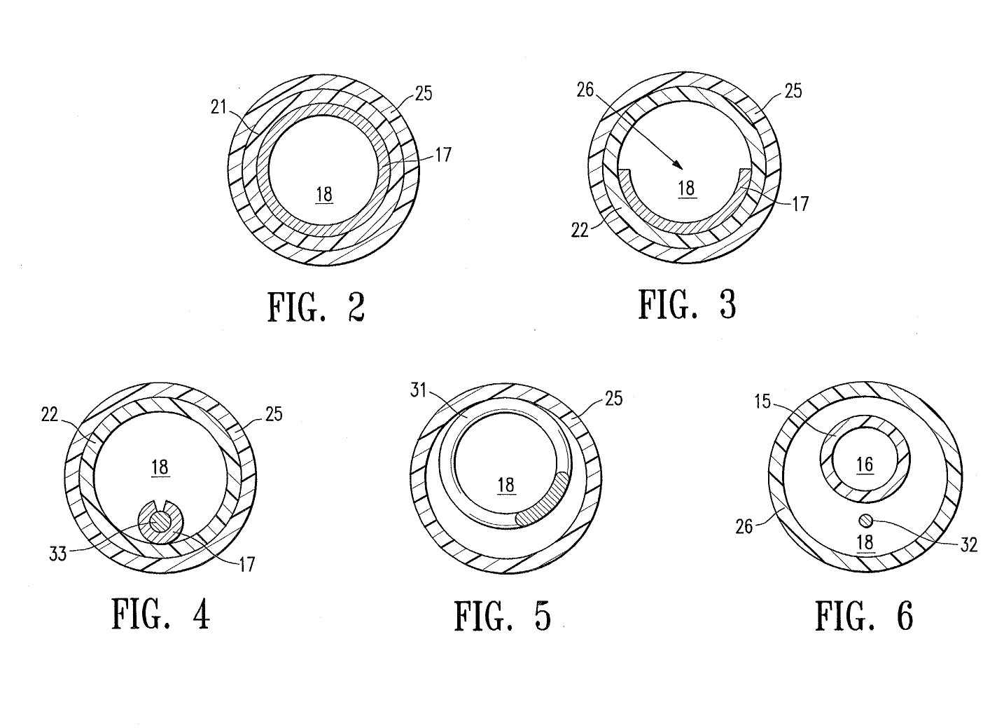

FIGS. 1-6 illustrate a rapid exchange type balloon catheter 10 embodying features of the invention. Catheter 10 generally comprises an elongated catheter shaft 11 having a proximal end, a distal end, a proximal shaft section 12, and a distal shaft section 13. Distal shaft section 13 has an outer tubular member 14, and an inner tubular member 15 defining a guidewire lumen 16 adapted to slidingly receive a guidewire (not shown). Proximal shaft section 12 comprises a tubular member 17 defining a proximal portion of an inflation lumen 18, in fluid communication with a distal portion of the inflation lumen 18 defined by the outer tubular member 14 of the distal shaft section 13. An inflatable balloon 19 is disposed on the distal shaft section 13, and has a proximal skirt section sealingly secured to the distal end of outer tubular member 14 and a distal skirt section sealingly secured to the distal end of inner tubular member 15, so that its interior is in fluid communication with inflation lumen 18. An adapter 20 at the proximal end of the shaft is configured to direct inflation fluid into inflation lumen 18. Balloon 19 has an inflatable working length located between tapered sections of the balloon. FIG. 1 illustrates the balloon 19 in an unexpanded configuration prior to inflation. The distal end of catheter may be advanced to a desired region of a patient's body lumen in a conventional manner, and balloon 19 inflated to perform a procedure such as dilating a stenosis or implanting a stent (not shown). FIGS. 2-6, illustrate transverse cross sections of the catheter 10 of FIG. 1, taken along lines 2-2, 3-3, 4-4, 5-5, and 6-6, respectively.

As a rapid exchange catheter, the shaft has a guidewire proximal port 23 in the distal shaft section 13 in fluid communication with the guidewire lumen 16 in the inner tubular member 15. A guidewire distal port 24 is at the distal end of the inner tubular member 15 and is in fluid communication with the guidewire lumen 16. The guidewire proximal port 23 allows a guidewire to exit the catheter 10 proximally therefrom and extend alongside and exteriorly of the proximal shaft section 12 to the proximal end of the catheter 10. In the embodiment of FIG. 1, the distal shaft section 13 outer tubular member 14 comprises a first outer tubular member 25, and second outer tubular member 26 with a proximal end secured to the distal end of the first outer tubular member 25. The guidewire proximal port 23 is in a side wall of the first outer tubular member 25. The second outer tubular member 26 is typically more flexible than the first outer tubular member 25, as, for example, by being formed of a lower Shore durometer polymer. In the embodiment of FIG. 1, at least a section of the inner tubular member 15 is coaxially disposed within the outer tubular member 14, to define an annular distal portion of inflation lumen 18 therebetween. Although not illustrated in FIG. 1, it should be understood that a portion of a proximal section of the inner tubular member 15 may be fused to a section of the first outer tubular member 25 distal to the guidewire proximal port 23, so that the distal shaft section 13 has a dual lumen portion with the inflation lumen 18 and the guidewire receiving lumen 16 extending therein in a side by side and a parallel relationship.

The tubular member 17 of the proximal shaft section 12 is preferably a high strength tubular member formed of metal or other high strength material with an exterior polymeric jacket 21. In the embodiment illustrated in FIG. 1, a polymeric reinforcing tubular member 22 is secured to the distal end of the high strength tubular member 17, and extends distally thereof to improve kink resistance at the distal end of the high strength tubular member 17. The polymeric reinforcing tubular member 22 is preferably formed of a high strength polymer such as polyetheretherketone (PEEK), however a variety of-suitable polymers may be used including polyamide and reinforced polymers. The inflation lumen 18 within the proximal shaft section is defined by the high strength tubular member 17 and the polymeric reinforcing tubular member 22. The distal tip of the high strength tubular member 17 is tapered distally to smaller transverse dimensions. Specifically, in the embodiment of FIG. 1, the high strength tubular member 17 distal section has a cut-out region with a truncated wall section defining a port 26 extending along a part of the length of the high strength tubular member 17. The cut-out region is about 1 cm to about 4 cm, preferably about 1.2 cm to about 1.5 cm in length, defining longitudinally extending port 26.

A support member 30 has a proximal end within the distal end of the high strength tubular member 17 of the proximal shaft section 12. In the embodiment of FIG. 1, the support member 30 is formed of a solid metal wire with a circular transverse cross section. However, a variety of suitable configurations may be used including a ribbon with a flat transverse cross section, or an oval transverse cross section. The support member 30 is preferably formed of a stainless steel or NiTi alloy (NITINOL), and the stainless steel is typically fill hard or spring tempered. The wire or ribbon of support member 30 has a diameter of about 0.003 to about 0.010 inches (0.08 to about 0.25 mm), preferably about 0.005 inches (0.13 mm).

The support member 30 has a coiled section 31 proximal to the guidewire proximal port 23. The support member has a distal section 32 extending across the guidewire proximal port 23, from a location proximal to the guidewire proximal port 23 to a location in the second outer tubular member 26 distal to the guidewire proximal port 23, to provide kink resistance at the rapid exchange junction. The proximal section 33 is in part within the distal end of the high strength tubular member 17. In one embodiment, proximal and distal sections 33 and 32 of support member 30 are not coiled, and in the embodiment of FIG. 1 are substantially straight and axially aligned with the distal shaft section 13. The noncoiled distal section 32 is located distal to the coiled section 31 and has a distal portion which tapers distally to smaller transverse dimensions, providing a gradually increasing flexibility. In an alternative embodiment, distal section is not tapered. Preferably, the distal section 32 has a proximal portion which is located between the tapered distal portion and the coiled section 31, which has a constant diameter, and which is about 5% to about 50% of the length of the distal section 32. Thus, the portion of the distal section 32 closest to the coiled section 31 is preferably not tapered. The tapered distal portion is preferably a gradual, constant taper, although one or more stepped tapers may alternatively be provided. In the embodiment of FIG. 1, proximal section 33 of support member 30 extends along port 26 in the distal tapered section of the high strength tubular member 17. At least a section of the distal tapered section of the high strength tubular member 17 has a larger inner diameter than the outer diameter of the proximal section 33 of the support member 30, so that port 26 is not occluded by the support member 30.

A section of the support member is wound one or more times to form the coiled section 31. Preferably, the coiled section 31 has tightly packed or stacked coils which are not spaced apart, in order to provide enhanced pushability to the catheter shaft. However, in an alternative embodiment (not shown), the coils are spaced apart in a loosely coiled configuration, and the pitch or spacing of the coils may vary along the length of the coiled section. In one embodiment, the support member 30 has about 200 to about 600 stacked coils in a tightly wound configuration, and more specifically about 300 to about 400 stacked coil turns. The coiled section 31 has an outer diameter configured to fit within the inner lumen 18 of a proximal portion of the distal shaft section 13, and specifically of about 0.015 to about 0.040 inches (about 0.38 to about 1.0 mm), and preferably about 0.026 inches (0.66 mm), for a 0.028 inch (0.71 mm) inner diameter tubular member 25. The coiled section 31 has an inner diameter configured to define a portion of the inflation lumen 18 and provide fast inflation/deflation, and specifically of about 0.01 to about 0.03 inches (about 0.25 to about 0.76 mm), and preferably about 0.016 inches (0.40 mm) for a 0.028 inch (0.71 mm) inner diameter shaft section. In one embodiment the proximal section 33 is about 0.1 to about 2 cm, preferably about 1 cm in length, the coiled section 31 is about 1.0 to about 10 cm, preferably about 5 cm in length, and the distal section 32 is about 3 to about 10 cm, preferably about 5 cm in length, for a support member 30 having a length of about 4 to about 22 cm.

During formation and assembly of the catheter 10, a portion of the high strength tubular member 17 distal section is cut off, preferably at a slant, leaving a tapered distal end portion of the high strength tubular member 17 with a height of about 0.003 to about 0.018 inch (0.08 to about 0.46 mm) at the distal tip, and thereby forming distal opening 26. The proximal section 33 of the support member is positioned within the tapered distal end portion of the high strength tubular member 17. The tapered distal end portion of the high strength tubular member 17 is wrapped, as for example by crimping, around the support member proximal section 33, although it may not extend around the entire circumference of the support member proximal section 33, as illustrated in FIG. 4. In the embodiment illustrated in FIG. 4, the support member proximal section 33 occludes the high strength tubular member lumen distal to the port 26. However, in an alternative embodiment (not shown), the support member proximal section 33 has a diameter which is sufficiently small so that the support member 30 does not occlude the lumen at the distal end of the high strength tubular member 17.

In one embodiment, the support member proximal section 33 is bonded to the high strength tubular member 17, as for example by adhesive bonding, or by soldering or welding, or otherwise secured as for example by crimping. In an alternative embodiment (not shown), the support member is secured to a portion of the shaft other than the high strength tubular member 17 and is not fixedly secured to the high strength tubular member 17. The coiled section 31 and the distal noncoiled section 32 are typically not bonded to the polymeric tubular members of the distal shaft section 13, to allow for flexing around curves in the patient's vessels for improved flexibility and kink resistance.

In a presently preferred embodiment, the distal shaft section 13 outer tubular member 14, and specifically the first outer tubular member 25, is formed of a polyamide material such as Nylon, which is compatible with a polyamidepolymeric material such as polyether block amide (PEBAX) forming the second outer tubular member 26 of the distal shaft section 13 and a polyamide material such as Nylon forming the exterior jacket 21 on the high strength proximal tubular member 17, to allow for fusion bonding the sections together. However, a variety of polymeric materials and suitable methods of bonding can be used including adhesive bonding. Additionally, although lap joints are illustrated in FIG. 1 between the tubular members, a variety of suitable joints may be used including a butt joint.

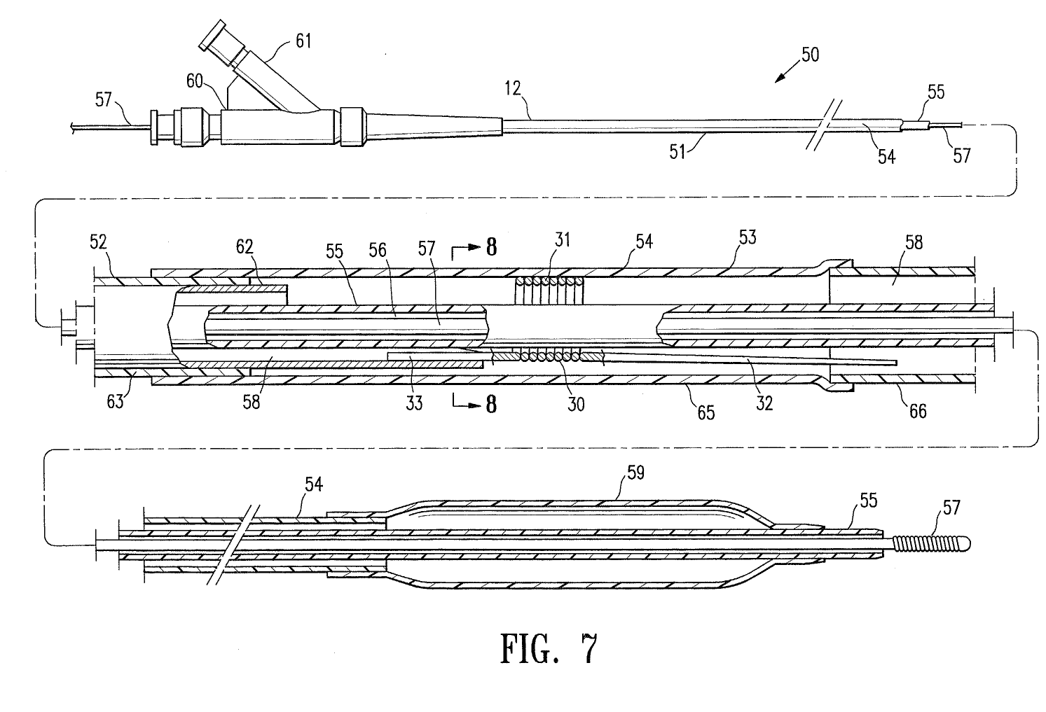

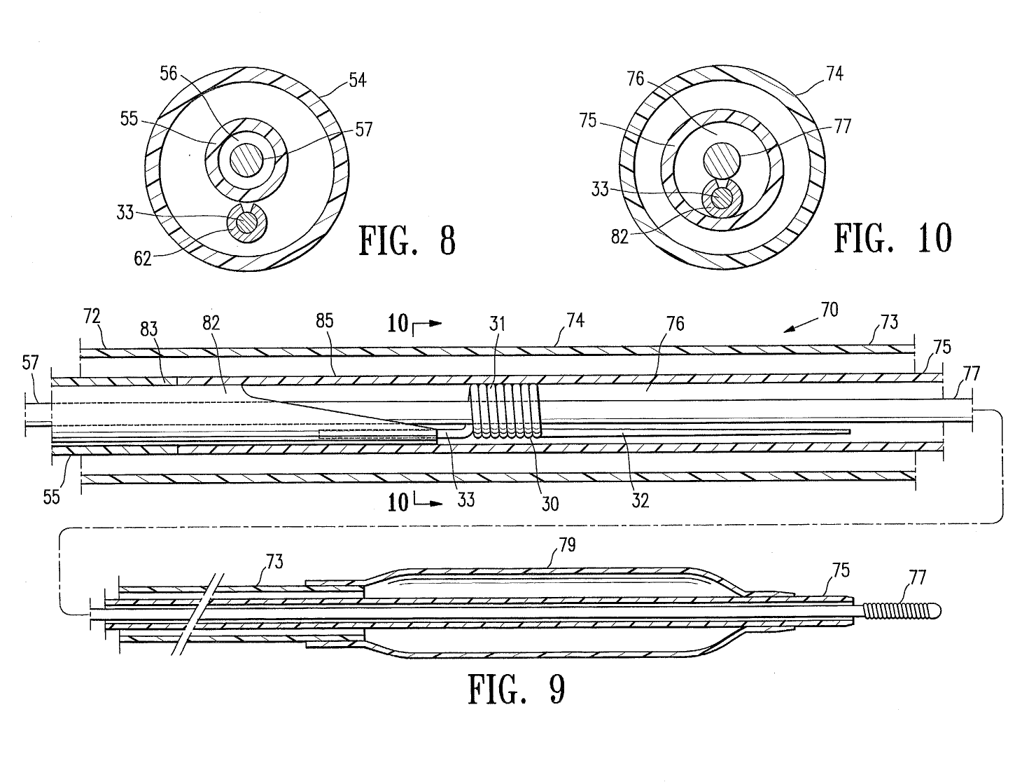

FIGS. 7-8 illustrate an alternative embodiment of the invention, in which balloon catheter 50 is an over-the-wire catheter having a support member 30 within the distal end of a high strength tubular member forming a proximal section of an outer tubular member. Catheter 50 generally comprises an elongated catheter shaft 51 having a proximal end, a distal end, a proximal shaft section 52, a distal shaft section 53, an outer tubular member 54, and an inner tubular member 55. Inner tubular member 55 extends to the proximal end of the catheter 50 and defines a guidewire lumen 56 adapted to slidingly receive a guidewire 57. Inflation lumen 58 is defined by the outer tubular member 54. An inflatable balloon 59 is disposed on the distal shaft section 53, having a proximal skirt section sealingly secured to the distal end of outer tubular member 54, and a distal skirt section sealingly secured to the distal end of inner tubular member 55, so that its interior is in fluid communication with inflation lumen 58. An adapter 60 at the proximal end of the shaft is configured to provide access to guidewire lumen 56, and to direct inflation fluid through arm 61 into inflation lumen 58. A high strength tubular member 62 formed of metal with an exterior polymeric jacket 63 forms a proximal section of outer tubular member 54 and defines a proximal portion of inflation lumen 58. Thus, similar to the embodiment of FIG. 1, the catheter 50 proximal shaft section 52 comprises high strength tubular member 62 with support member 30 within the distal end thereof. A distal section of the outer tubular member 54 is more flexible than the high strength tubular member 62, and is formed by a first outer tubular member 65, and a second outer tubular member 66 with a proximal end secured to the distal end of the first outer tubular member 65.

FIGS. 9-10 illustrate an alternative embodiment of the invention, in which balloon catheter 70 is an over-the-wire catheter having support member 30 within the distal end of a high strength tubular member forming a proximal section of an inner tubular member. Similar to the embodiment of FIG. 7, catheter 70 generally comprises an elongated catheter shaft having a proximal end, a distal end, a proximal shaft section 72, a distal shaft section 73, an outer tubular member 74, an inner tubular member 75, and inflatable balloon 79. Inner tubular member 75 defines a guidewire lumen 76 adapted to slidingly receive a guidewire 77. A high strength tubular member 82 formed of metal with an exterior polymeric jacket 83 forms a proximal section of inner tubular member 75 and defines a proximal portion of guidewire lumen 76. Thus, similar to the embodiment of FIG. 1, the catheter 70 proximal shaft section 72 comprises high strength tubular member 82 with support member 30 within the distal end thereof. A distal section of inner tubular member 75 is formed by tubular member 85 which is more flexible than the high strength tubular member 82.

When the catheter of the invention is used in an angioplasty procedure, the balloon catheter of the invention is advanced over the guidewire until the balloon is properly positioned across the stenosis. The balloon can be inflated in a conventional manner by introducing inflation fluid through the inflation lumen. After one or more inflations, the balloon is deflated and the catheter removed from the patient. A similar procedure is used when the balloon has a stent (not shown) mounted thereon for implanting the stent in the body lumen.

To the extent not previously discussed herein, the various catheter components may be formed and joined by conventional materials and methods. For example, inner tubular member 15 and outer tubular member 14 can be formed by conventional techniques, such as by extruding and necking materials found useful in intravascular catheters such as polyethyene, polyvinyl chloride, polyesters, polyamides, polyimides, polyurethanes, and composite materials.

The length of the dilation catheter 10/50/70 is generally about 108 to about 200 centimeters, preferably about 137 to about 145 centimeters, and typically about 140 centimeters for PTCA. The outer tubular member 14/54/74 distal section has an outer diameter (OD) of about 0.028 to about 0.036 inch (0.70 -0.91 mm), and an inner diameter (ID) of about 0.024 to about 0.035 inch (0.60-0.89 mm), and proximal tubular member 17 or the outer tubular member 54/74 proximal section has an OD of about 0.017 to about 0.034 inch (0.43-0.87 mm), and an ID of about 0.012 to about 0.022 inch (0.30-0.56 mm). The inner tubular member 15/55/75 has an OD of about 0.017 to about 0.026 inch (0.43-0.66 mm), and an ID of about 0.015 to about 0.018 inch (0.38-0.46 mm) depending on the diameter of the guidewire to be used with the catheter. The balloon 19 is typically about 14 to about 46 mm in length, with an inflated working diameter of about 8 to about 40 mm.

While the present invention has been described herein in terms of certain preferred embodiments, those skilled in the art will recognize that modifications and improvements may be made without departing from the scope of the invention. For example, while the catheter illustrated in the figure has coaxial inner and outer tubular members, other conventional catheter shaft configurations can be used along at least a section of the catheter, such as side-by-side, dual lumen configurations. Additionally, the polymeric reinforcing tubular member 22 illustrated in the embodiment of FIG. 1 may be provided in the embodiments of FIGS. 7 and 9. Moreover, while individual features of one embodiment of the invention may be discussed or shown in the drawings of the one embodiment and not in other embodiments, it should be apparent that individual features of one embodiment may be combined with one or more features of another embodiment or features from a plurality of embodiments.

What is claimed is:

1. An elongated intracorporeal device, comprising:
a) an elongated shaft having a proximal shaft section and a distal shaft section; and
b) a support member secured to the shaft, having a proximal end within the proximal shaft section, a distal end within the distal shaft section, and having at least one section which is coiled and at least one section which is not coiled.

2. A balloon catheter, comprising:
a) an elongated shaft with an inflation lumen, a guidewire receiving lumen, a proximal shaft section having proximal and distal ends and a proximal portion of the inflation lumen, and a distal shaft section having proximal and distal ends and a distal portion of the inflation lumen in fluid communication with the proximal portion of the inflation lumen, and having at least part of the guidewire receiving lumen extending within the distal shaft section to a guidewire distal port in the distal end thereof,
b) an inflatable balloon on the distal shaft section having an interior in fluid communication with the inflation lumen; and
c) a support member secured to the shaft having a proximal end within the proximal shaft section, a distal end within the distal shaft section, and having at least one section which is coiled and at least one section which is not coiled.

3. The balloon catheter of claim 2 wherein the support member has a noncoiled proximal section at least in part within the proximal shaft section, and the coiled section is distal to the noncoiled proximal section.

4. The balloon catheter of claim 3 wherein the support member has a distal noncoiled section distal to the coiled section, so that the coiled section is located between the proximal and distal noncoiled sections.

5. The balloon catheter of claim 4 wherein the distal noncoiled section has a distal portion which tapers distally to smaller transverse dimensions.

6. The balloon catheter of claim 4 wherein the support member has a length of about 4 to about 22 cm.

7. The balloon catheter of claim 6 wherein the coiled section of the support member has a length of about 1 to about 10 cm.

8. The balloon catheter of claim 2 wherein the coiled section of the support member comprises stacked coils.

9. The balloon catheter of claim 2 wherein the support member is a metal wire.

10. The balloon catheter of claim 2 wherein the support member proximal end is secured to the distal end of the proximal shaft section.

11. The balloon catheter of claim 2 wherein the proximal shaft section comprises a metallic tubular member.

12. The balloon catheter of claim 11 wherein the distal end of the metallic tubular member of the proximal shaft section has a cut-out region with a truncated wall section defining a port extending along a part of the length of the metallic tubular member.

13. The balloon catheter of claim 12 wherein the distal end of the metallic tubular member extends around the proximal end of the support member.

14. The balloon catheter of claim 2 wherein the distal shaft section has at least a distal portion of an inner tubular member which defines at least a distal portion of the guidewire receiving lumen, and has an outer tubular member disposed around the inner tubular member along a portion of the length of the inner tubular member, so that the inflation lumen within the distal shaft section is between the outer tubular member and the inner tubular member.

15. The balloon catheter of claim 14 wherein the shaft has a guidewire proximal port in the distal shaft section in fluid communication with the inner tubular member guidewire receiving lumen.

16. The balloon catheter of claim 15 wherein the support member distal end is in the inflation lumen and is distal to the guidewire proximal port, and the support member has a proximal and a distal noncoiled section on either end of the coiled section.

17. The balloon catheter of claim 16 wherein the distal noncoiled section extends from a location proximal to the guidewire proximal port to a location distal to the guidewire proximal port.

18. The balloon catheter of claim 15 wherein the support member coiled section is not secured to the outer tubular member of the distal shaft section.

19. The balloon catheter of claim 15 wherein the support member coiled section is secured to the outer tubular member of the distal shaft section.

20. The balloon catheter of claim 15 including a polymeric tubular reinforcing member having a proximal end surrounding the distal end of the metallic tubular member of the proximal shaft section, and a distal end extending within the inflation lumen in the distal shaft section to a location proximal to the guidewire proximal port, with a part of the inflation lumen extending within the polymeric tubular reinforcing member.

21. The balloon catheter of claim 15 wherein the distal shaft section comprises a first outer tubular member, and second outer tubular member with a proximal end secured to the distal end of the first outer tubular member, and the guidewire proximal port is in the first outer tubular member.

22. The balloon catheter of claim 14 wherein the inner tubular member extends within the metallic tubular member of the proximal shaft section and to the proximal end of the catheter.

23. The balloon catheter of claim 14 wherein the outer tubular member extends to the proximal end of the catheter, and the shaft has a guidewire proximal port at the proximal end of the catheter.

24. The balloon catheter of claim 23 wherein the metallic tubular member of the proximal shaft section is within a proximal portion of the outer tubular member, and has a distal end secured to the proximal end of the inner tubular member of the distal shaft section, and defines a proximal portion of the guidewire receiving lumen, so that the support member is within the guidewire receiving lumen.

25. A rapid exchange type balloon catheter, comprising:
a) an elongated shaft with an inflation lumen, a guidewire receiving lumen, a proximal shaft section having proximal and distal ends and a proximal portion of the inflation lumen, and a distal shaft section having proximal and distal ends and a distal portion of the inflation lumen in fluid communication with the proximal portion of the inflation lumen, and having a guidewire proximal port, a guidewire distal port in the distal end thereof, and the guidewire receiving lumen extending there between within the distal shaft section;
b) an inflatable balloon on the distal shaft section having an interior in fluid communication with the inflation lumen; and
c) a support member secured to the shaft and extending from a location proximal to the guidewire proximal port to a location distal to the guidewire proximal port, and having a proximal end within the proximal shaft section, a distal end within the distal shaft section, and at least a section which is coiled.

26. The balloon catheter of claim 25 wherein the support member has a noncoiled proximal section at least in part within the proximal shaft section, and a noncoiled distal section, and the coiled section is located between the proximal and distal noncoiled sections.

27. The balloon catheter of claim 25 wherein the coiled section is proximal to the guidewire proximal port.

28. The balloon catheter of claim 25 wherein the distal noncoiled section extends distal to the guidewire proximal port.
